(19) United States
(12) Patent Application Publication
(10) Pub. No.: US 2009/0263889 A1
(43) Pub. Date: Oct. 22, 2009

(54) Method For Increasing Yield of Biomass of and/or Components of Biomass From Marine Microorganisms

(75) Inventor: Mogens Wumpelmann, Copenhagen O (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/493,505

(22) Filed: Jun. 29, 2009

Related U.S. Application Data
(63) Continuation of application No. 10/570,398, filed on Mar. 2, 2006.
(60) Provisional application No. 60/499,938, filed on Sep. 3, 2003.

(30) Foreign Application Priority Data
Sep. 1, 2003 (DK) PA 2003 01237

(51) Int. Cl. C12N 1/12; C12N 1/10

(52) U.S. Cl. 435/257.1; 435/258.1

(57) ABSTRACT

The present invention provides an optimized method of continuously culturing an auxotrophic marine microorganism in a fermentor under aerobic conditions at Y g/l of cell dry matter, CDM, wherein Y is in the range from 100-300 g/l, comprising culturing said auxotrophic marine microorganism in a culture medium comprising a carbon source, gradually added, in an amount of (Y×h) gram per litre of culture broth, wherein h is in the range from 1.1-3.0, and with a residence time of 20-100 h. The method maintains a high productivity of cellular lipids, especially polyenoic acids.

### CROSS-REFERENCE TO RELATED APPLICATIONS

This application is a continuation of U.S. application Ser. No. 10/570,398 filed Feb. 27, 2006 which is a 35 U.S.C. 371 national application of PCT/DK2004/000561 filed Aug. 24, 2004, which claims priority or the benefit under 35 U.S.C. 119 of Danish application no. PA 2003 01237 filed Sep. 1, 2003 and U.S. provisional application No. 60/499,938 filed Sep. 3, 2003, the contents of which are fully incorporated herein by reference.

### FIELD OF INVENTION

The present invention relates to a method of culturing a marine microorganism under aerobic conditions, wherein 100-300 g/l of cell dry matter, CDM, is produced in 20-100 hours employing a continuous fermentation process.

### BACKGROUND OF THE INVENTION

In the industrial production of biomass or components constituting a significant part of the biomass by batch, fed batch or continuous cultivation of microorganisms, it is desirable to achieve the highest possible biomass productivity. Further, a fermentation process constituting essentially a continuous operation has the advantage of low man-power requirements as well as potentially low requirements for process control. Continuous fermentation processes rely on strains employed being sufficiently stable, and if such strains are available, the employment of continuous fermentation processes provides potentially a manufacturing process allowing for higher degrees of homogeneity with regard to the overall cultivation broth characteristics including product concentration and product recoverability to be achieved.

U.S. Pat. No. 5,244,921 describes a method for producing eicosapentaenoic acid (EPA) in commercially viable yields from diatoms such as Nitzschia alba, resulting in yields of less than 70 g CDM/I in 60 hours.

U.S. Pat. No. 5,711,983 relates to a method for producing docosahexaenoic acid (DHA) in commercially viable yields from marine dinoflagellates including Crypthecodinium sp. Yields are reported in the range of 23 g CDM/I in 75 hours and 33 g CDM/I in 160 hours.

EP 0823475 A1 relates to the production of DHA and DPA from the Schizochytrium genus SR21. The resulting yields are reported to be at the most 60 g CDM/I in 150 hours.

U.S. Pat. No. 5,518,918 relates to microfloral biomass comprising a microorganism selected from the group consisting of Thraustochytrium and Schizochytrium. The obtained CDM is less than 8 g/l.

WO 01/04338 relates to a method of culturing a microorganism, Crypthecodinium cohnii, for the synthesis of a polyunsaturated fatty acid. The obtained yields are less than 46 g CDM/I in 140 hours.

U.S. Pat. No. 6,582,941 relates to a Schizochytrium strain. The obtained yields are less than 60 g CDM/I in 120 h.

WO 01/54510 relates to eukaryotic microorganisms, and in particular to micro algae of the order Thraustochytriads, cultivated in fed batch fermentation processes, and emphasizing the importance of separating the overall fermentation process into two phases: one for initial build-up of biomass and one phase allowing for the accumulation of polyenoic fatty acids to occur at conditions of specified nutrient-limitation and low oxygen tension. More than 100 g/l cell dry matter containing at least 20% w/w lipids is achieved while the productivity of DHA (omega-3 C22:6, docosahexaenoic acid) can be higher than 0.3 g/l/h at fed batch fermentation processes. However, likely due to the complex nature of the fermentation process involved, the DHA-productivity was demonstrated to vary by a factor of ˜2 within 31 identical fermentation batches carried out (see Example 4). WO 01/54510 also demonstrates that yields of up to 20 g/l cell dry matter may be achieved when using a continuous fermentation process (see Example 9).

Methods for simplifying the fermentation process for cultivating oleagineous, polyenoic acid producing micro algae while maintaining high polyenoic acid productivities are therefore still needed.

### SUMMARY OF THE INVENTION

The present invention provides such an improved method for cultivation of auxotrophic marine micro organisms resulting in very high biomass productivities, wherein yields of 100-300 g/l of cell dry matter can be harvested from a continuously operated fermentor for which the culture broth residence time is in the range of 20-100 hours while maintaining a lipid content of around 0.5 g lipid/g biomass dry matter and a polyenoic acid productivity of at least 0.2 g DHA/I/h.

Given prior art it is most surprising, that such high polyenoic acid productivities can be achieved without decoupling cell growth and polyenoic acid production.

In a first aspect the present invention relates to a method of continuously culturing an auxotrophic marine microorganism in a fermentor under aerobic conditions at Y g/l of cell dry matter, CDM, wherein Y is in the range from 100-300 g/l, comprising culturing said auxotrophic marine microorganism in a culture medium comprising a carbon source, gradually added, in an amount of (Y×h) gram per litre of culture broth, wherein h is in the range from 1.1-3.0, and with a residence time of 20-150 h, in particular with a residence time of 20-100 h.

Stating the range of h it is understood that the amount of carbon source is given as free of any associated water. In the following paragraphs it is understood that amounts of nitrogen source is given as amount of nitrogen.

### DETAILED DESCRIPTION OF THE INVENTION

It is well known that microorganisms need a carbon source in order to grow. Also the concentration of the carbon source in the medium is important for the final yield of cell dry matter.

Surprisingly we have found that by increasing the concentration of the carbon source in the medium fed to a continuously operating fermentation process it is possible to obtain yields (Y) expressed as cell dry matter, CDM, in the order of 100-300 g/l, said amount of biomass being produced in less than 100 h, when culturing a marine microorganism in a culture medium in which either the carbon source or the nitrogen source is limiting for biomass formation while maintaining high lipid and high polyenoic productivities.

The carbon source should be added in an amount of Y×h gram per litre of culture broth, wherein h is in the range from 1.1 to 3.0, preferably in the range from 1.1-2.5, even more preferably in the range of from 1.2-2.0.

Nitrogen, in the form of, e.g., casamino acids and/or (NH₄)₂SO₃, should be made available in amounts that are from 0.002 to 0.2 times the amount of the carbon source (Y×h×f), preferably in amounts that are from 0.004 to 0.1 times the amount of the carbon source, even more preferably in amounts that are from 0.01 to 0.04 times the amount of the carbon source.

In one embodiment the present invention therefore relates to a method of continuously culturing an auxotrophic marine microorganism under aerobic conditions, wherein Y g/l of cell dry matter, CDM, at a given point can be harvested from the fermentor within 20-100 hours, wherein Y is comprised in the range from 100-300 g/l, comprising culturing said marine microorganism in a culture medium comprising:

i) a carbon source, continuously added, in an amount of (Y×h) gram per litre of culture broth, wherein h is comprised in the range from 1.1-3.0; and
ii) a nitrogen source, continuously added, in an amount of from Y×h×f, wherein f is comprised in the range from 0.002 to 0.2.

Also additional components such as salts, minerals and vitamins required for biomass formation need to be supplied to the microorganism by the addition of these components to the growth medium. The components should be added in such amounts that further addition of these components will have no significant effect on biomass concentrations achieved.

### Design of the Culturing Method

Many different designs of the culturing method can be applied.

In a preferred embodiment, not in any way limiting the scope of the present invention, the culturing method is a continuous fermentation process comprising 3 cultivation steps:
a) an initial batch process, followed by
b) a fed batch process, followed by
c) a continuous process, wherein a medium is continuously added at a constant feed rate and in which, the culture broth is continuously removed in such a way, that the total broth weight is maintained, so we also claim:

A method of continuously culturing an auxotrophic marine microorganism in a fermentor under aerobic conditions at Y g/l of cell dry matter, CDM, wherein Y is in the range from 100-300 g/l, comprising culturing said auxotrophic marine microorganism in a culture medium comprising a carbon source, gradually added, in an amount of (Y×h) gram per litre of culture broth, wherein h is in the range from 1.1-3.0, and with a residence time of 20-100 h, wherein the continuous fermentation process comprises 3 cultivation steps:
a) an initial batch process, followed by
b) a fed batch process, followed by
c) a continuous process.

Phase a) and b) serves primarily one objective, that is to allow the biomass concentration to reach levels >50% of biomass concentrations reached upon achieving a steady state status in phase c), this allowing for harvest of biomass from phase c) initially to occur at concentrations close to the steady state biomass concentration eventually achieved in phase c). The composition of the medium employed for the initial batch phase as well as for the fed batch phase should reflect this objective.

A shift from phase a) to phase b) should occur before the carbon source in the phase a) medium becomes exhausted.

A shift from phase b) to phase c) should occur

i) at a time suitable for the collective objective for phase a) and b) stated above to be reached and
ii) at a time dependent on the carbon and nitrogen source concentration in the feed medium used in phase b), as well as on the carbon and nitrogen source concentration in the batch medium of phase a).

It should be understood that it is the characteristics of the continuous process when entering into a steady state status that constitutes the description of the overall process with regard to the biomass productivity achieved and specifications of media used.

For someone skilled in the art it is obvious that continuous fermentation processes usually employ a constant culture broth residence time. However, for someone skilled in the art it is also known that varying the residence time can improve the overall performance of continuous fermentation processes and such variation is within the scope of this invention, so we claim the following two processes:

A method according to present invention, wherein the residence time of the culture broth in the continuous cultivation process is maintained constant and in the range of 20-100 h; and a method according to present invention, wherein the residence time of the culture broth in the continuous cultivation process is varied within the range of 20-100 h.

The amount of nitrogen can also be varied and should correspond to the amount of carbon source in such a way that the total concentration of organic and inorganic nitrogen, N_{konc}., is Y×h×f.

When culturing marine microorganisms according to the present invention it is possible to obtain a biomass productivity in the form of CDM that can be harvested from the fermentor in the range of 0.67 to 15 g cell dry matter per litre culture medium per hour while maintaining a lipid content of around 0.5 g/g biomass dry matter and while maintaining high polyenoic acid productivities of at least 0.20 g DHA/I/h, preferably of at least 0.25 g DHA/I/h, more preferably of at least 0.30 g DHA/I/h, most preferably of at least 0.35 g DHA/I/h.

In a preferred embodiment the method according to the invention may produce polyenoic acid in a concentration of 0.20-0.40 g DHA/I/h, preferably in a concentration of 0.25-0.4 g DHA/I/h, more preferably in a concentration of 0.30-0.40 g DHA/I/h, most preferably in a concentration of 0.35-0.40 g DHA/I/h.

The fermentation according to the present invention is in one embodiment carried out at levels of dissolved oxygen above 10% of saturation. However, carrying out the fermentation at lower levels is according to WO 01/54510 likely to enhance the productivity in polyenoic fatty acids formation even further. The advantage of employing continuous fermentation processes versus fed batch fermentation processes when one objective of fermentation control is to maintain the level of dissolved oxygen at low levels is—for someone skilled in the art—obvious, since such control in continuous processes can be achieved simply by adjusting the aeration and the agitation rates at fixed levels while such control in fed batch processes must rely on accurate measurements of dissolved oxygen to be carried out throughout the fermentation process. Further, such accurate measurement of dissolved oxygen is subject to failure.

Thus, we also claim:

A method of continuously culturing an auxotrophic marine microorganism in a fermentor under aerobic conditions at Y g/l of cell dry matter, CDM, wherein Y is in the range from 100-300 g/l, comprising culturing said auxotrophic marine microorganism in a culture medium comprising a carbon source, gradually added, in an amount of (Y×h) gram per litre of culture broth, wherein h is in the range from 1.1-3.0, and with a residence time of 20-100 h, wherein the continuous fermentation process comprises 3 cultivation steps:
a) an initial batch process, followed by
b) a fed batch process, followed by
c) a continuous process
   and wherein the level of dissolved oxygen tension in step c) is maintained below 10% of saturation, preferably below 5% of saturation, more preferably below 1% of saturation.

The fermentation according to the present invention is in one embodiment carried out at a cultivation temperature in the range from 20 to 35° C., particularly in the range from 25 to 30° C.

The pH in the culturing medium should be comprised in the range from 3.0 to 9.0, particularly in the range from 5.0 to 7.5.

### Auxotrophic Marine Microorganisms

A preferred auxotrophic marine microorganism according to the invention is an algae, in particular a micro algae or an algae-like microorganism, preferably a member of the Stramenopiles group, more preferably a Hamatores sp, a Proteromonads sp, a Opalines sp., a Developayella sp, a Diplophrys sp, a Labrinthulids sp, a Thraustochytrids sp, a Biosecids sp, an Oomycetes sp, a Hypochytridiomycetes sp, a Commation sp, a Reticulosphaera sp, a Pelagomonas sp, a Pelagococcus sp, an Ollicola sp, an Aureococcus sp, a Parmales sp, a Diatoms sp, a Xanthophytes sp, a Phaeophytes sp (brown algae), a Eustigmatophytes sp, a Raphidophytes sp, a Synurids sp, an Axodines sp, a Chrysomeridales sp, a Sarcinochrysidales sp, a Hydrurales sp, a Hibberdiales sp, or a Chromulinales sp.

A specially preferred marine microorganism according to the invention is a Thraustochytrids sp, in particular a Schizochytrium sp or a Thraustochytrium sp. Most preferred is a Schizochytrium sp, in particular a S. limacinum sp, preferably strain SR21 (FERM BP-5034).

### The Lipid Content

The process of the present invention may be used to produce a variety of lipid compounds, in particular unsaturated lipids, preferably polyunsaturated lipids (i.e., lipids containing at least 2 unsaturated carbon-carbon bonds, e.g., double bonds), and more preferably highly unsaturated lipids (i.e., lipids containing 4 or more unsaturated carbon-carbon bonds) such as omega-3 and/or omega-6 polyunsaturated fatty acids, including docosahexaenoic acid (i.e., DHA); and other naturally occurring unsaturated, polyunsaturated and highly unsaturated compounds. As used herein, the term “lipid” includes phospholipids; free fatty acids; esters of fatty acids; triacylglycerols; sterols and sterol esters; carotenoids; xanthophylls (e.g., oxycarotenoids); hydrocarbons; isoprenoid-derived compounds and other lipids known in the art. In particular the method of the present invention is useful in producing polyenoic acid(s).

The lipid content in cell dry matter produced by the method according to the invention are components extractable by chloroform:methanol mixtures and constitutes at least 40% of the biomass produced, preferably at least 45% of the biomass produced, more preferably at least 50% of the biomass produced, even preferably at least 55% of the biomass produced. The chloroform:methanol ratio is in one embodiment 2:1 (v/v), preferably the chloroform:methanol ratio is in one embodiment 2:1(v/v), 0.1% butylhydroxy toluene.

Certain marine microorganisms, like, e.g., Thraustochytrids sp., produces desirable long chain polyunsaturated fatty acids (LC PUFA) like eicosapentaenoic acid (EPA) and docosahexaenoic acid (DHA).

Also, the clinical effects of enriching human diets with LC PUFA's have been extensively documented. LC PUFA's of particular interest is eicosapentaenoic acid (EPA) and docosahexaenoic acid (DHA). However, no consensus regarding the optimal ratio of EPA:DHA in diets for human adults has yet been reached and further, the ability of Thraustochytrids sp. to produce biomass, lipids and LC PUFA's highly efficient is not necessarily combined with the ability to produce the optimal ratio of EPA:DHA in one strain.

Thus, the aspect of modifying the characteristics of Thraustochytrids species with regard to biomass, lipid and LC PUFA productivity and/or with regard to the ratio of EPA:DHA produced in combination with an application of the present invention could be highly advantageous.

### EXAMPLES

### Example 1

### Cryopreservation of Schizochytrium limacinum, SR21 (FERM BP-5034)

The culture, received from the “National Institute of Bioscience and Human-Technology, Agency of Industrial Science and Technology, Japan” culture collection on agar, was transferred to a shake flask by suspending the cells on agar in “½TM” (described below). The shake flask (500 ml conical with 100 ml medium “OMEPRK_A” (described below)+10 ml cells in suspension) was incubated at 28° C. and 150 rpm in a Unitron, Infors AG thermostatically controlled rotary shaker for 25 h. 25 ml heat sterilised glycerol was added to the shake flask. After 40 min of incubation at room temperature aliquots of 1 ml were transferred to cryotubes.

Cryotubes (40 pc.) were slowly frozen by incubating the cryotubes in a flamingo-box (20×20 cm w/4 cm flamingo walls, lid and bottom) at −20° C. for 24 h and then transferring the cryotubes to a −80° C. freezer.

Cryotubes were maintained on stock at −80° C. until used.

### Media Used for Cultivation

“OmePRK_A”:

| | | |
|---|---|---|
| Tropic Marin ® (Article 10135) | 16.7 | g |
| KH₂PO₄: | 5 | g |
| Casamino acids, vitamin free: | 3 | g |
| “MikroPM” (described below): | 20 | ml |
| “VitaPM” (described below): | 20 | ml |

were all mixed.

pH was adjusted to 7.0 with NaOH/HCl.

Volume was adjusted to 900 ml with tap water.

Heat sterilisation was carried out at 121° C. for 20 min.

33 g Glucose 1H₂O in 100 ml, sterile filtered through 0.25 mikron filters were finally added. 100 ml was aseptically transferred to an empty, heat sterilised 500 ml conical shake flask.

“½TM”:

| | |
|---|---|
| Tropic Marin ®: | 16.7 g/l |

Solubilised in tap water.

Heat sterilisation was carried out at 121° C. for 20 min.

“MikroPM”:

| | |
|---|---|
| MnSO₄•1H₂O: | 0.98 g |
| FeSO₄•7H₂O: | 3.93 g |
| CuSO₄•5H₂O: | 0.39 g |
| ZnCl₂: | 0.39 g |
| Citric acid: | 19.6 g |

were all mixed, volume adjusted to 1.0 l with deionised water.

“VitaPM”:

| | | |
|---|---|---|
| Thiamin-dichloride: | 2.28 | g |
| Riboflavin: | 0.19 | g |
| Nicotinic acid: | 1.53 | g |
| Calcium D-pantothenat: | 1.9 | g |
| Pyridoxal HCl: | 0.38 | g |
| D-biotin: | 0.075 | g |
| Folic acid: | 0.19 | g |

were all mixed, volume adjusted to 1.0 l with deionised water.

### Example 2

### Propagation of the Schizochytrium limacinum strain SR21

The cells from 1 cryotube, thawn at room temperature, were transferred to and aseptically cultivated in 10 ml “OmePRK_A” medium contained in a 40 ml cylindrical glass and incubated for 24 h at 28° C. and 150 RPM (Unitron, Enfors AG).

The culture broth thus produced was transferred to and aseptically cultivated in 100 ml “OmePRK_A” medium contained in a 500 ml conical shake flask for 24 h at 28° C. and 150 RPM (Unitron, Enfors AG).

90 ml of the culture broth thus produced were used for inoculating a fermentor.

### Example 3

### Continuous Cultivation of the SR21 Strain at 30-35 h of Broth Residence Time

A 2 l glass/stainless steel fermentor of the Porton type was employed.

Outgrowth of the strain on 1.0 l medium “OME8” was allowed for 20 h maintaining
pH in the range 6.0-7.0 by the controlled addition of NaOH/H₃PO₄
temperature at 28° C.
agitation at 300 rpm linearly increasing to 400 rpm
aeration at 1.01/min
dissolved oxygen tension above 10% of saturation

At 20.1 h the fed batch feeding of the culture was initiated with medium “OME8a” (described below) at 0.057 g/min. A feed rate that was maintained until 100 h.

From 20 to 80 h agitation was increased linearly from 400 rpm to 500 rpm; other process parameters were maintained at previously stated values.

At 100 h a continuous cultivation mode was enforced by changing the feed medium to “OME17b” (described below), by increasing the feed rate to 0.5 g/min and by maintaining the total culture broth weight at 1000 g, allowing for culture broth to be removed from the fermentor by pumping. Further, agitation rate was increased at 100 h to 800 rpm. Foaming was controlled by manual addition of grape kernel oil.

As judged from measurements of OD (650 nm, 1 cm cuvette, 400 times dilution of broth in deionised water prior to measuring) and from the respiratory activity of the culture (% O₂ in the exhaust air as measured by an 1313 Fermentation Monitor from Innovo Air Tech. Instruments) steady state was achieved at ˜160 h.

At 190 h a sample of 50 ml was withdrawn, and centrifuged at 500 rpm and room temperature for 10 min in a Heraus Labofuge Ae; the pellet thus produced was gently washed with ˜35 ml “½TM”, centrifugation repeated and the pellet thus produced frozen at minus 80° C., and then freeze dried on a Hetosicc CD52-1 freeze dryer from Heto Lab Equipment.

A suspended solids dry weight concentration of 104.1 g/l could thus be determined. Since all media consisted of soluble components exclusively this FIGURE is taken as the cell dry weight concentration.

The residual glucose concentration was <<1 g/l from 25 h and onwards—as determined by using “Keto-diabur-test 5000” strips from ACCU-CHEK in conjunction with properly diluting samples.

In the present example Y=104.1 g/l and h=1.24 and f=0.021.

### “OME8”:

| | | |
|---|---|---|
| Tropic Marin ®: | 16.7 | g |
| KH₂PO₄: | 5 | g |
| Casamino acids, vitamin free: | 3 | g |
| (NH₄)₂SO₄: | 0.5 | g |
| “MikroPM”: | 20 | g |
| “VitaPM”: | 20 | g |

were all mixed, the pH adjusted to 6.5 with NaOH/H₃PO₄ and the volume adjusted to 700 ml.

Heat sterilisation of this medium was carried out at 121° C. for 40 min with the medium contained in the fermentor. After heat sterilisation and cooling to below 40° C., 33 g Glucose 1H₂O in tap water w/the volume adjusted to 300 ml prior to separate heat sterilisation at 121° C. for 40 min was added to the fermentor/medium thus producing “OME8”, ready for pH-adjustment in the fermentor to 6.5 and then inoculation. Tap water was used throughout.

“OME8a”:

All components are given in g/l.

All components—except for glucose—was heat sterilised together in 40% v/v of the final medium volume at 121° C. for 40 min after pH being adjusted to 5.0 with NaOH/H₃PO₄. Glucose was heat sterilised separately in 60% v/v of the final medium volume and then added to the other components after cooling to below 40° C.

Tap water was used throughout.

| | | |
|---|---|---|
| Tropic Marin ®: | 16.7 | g/l |
| KH₂PO₄: | 5 | g/l |
| “MikroPM”: | 20 | g/l |
| “VitaPM” | 20 | g/l |
| Casamino acids, vitamin free: | 45 | g/l |
| (NH₄)₂SO₄: | 7.5 | g/l |
| Glucose•1H₂O: | 495 | g/l |

“OME17b”:

All components are given in g/l.

All components—except for glucose—was heat sterilised together in 40% v/v of the final medium volume at 121° C. for 40 min after pH being adjusted to 5.0 with NaOH/H₃PO₄. Glucose was heat sterilised separately in 60% v/v of the final medium volume and then added to the other components after cooling to below 40° C.

Tap water was used throughout.

| | | |
|---|---|---|
| Tropic Marin ® | 16.7 | g/l |
| KH₂PO₄ | 5 | g/l |
| “MikroPM” | 20 | g/l |
| “VitaPM” | 20 | g/l |
| Casamino acids, vitamin free | 12.94 | g/l |
| (NH₄)₂SO₄ | 2.15 | g/l |
| Glucose•1H₂O | 142.3 | g/l |

### Example 4

### Continuous Cultivation of the SR21 strain at 60-70 h of Broth Residence Time

This cultivation was carried out as described in Example 3 with the following modifications:

When continuous cultivation mode was enforced at 100 h, then the feed flow rate was set at 0.25 g/min.

Further, at 190 h the feed medium was changed from “OME17b” to “OME17c” (described below).

At 285 h, 350 h, 450 h and 500 h a cell dry weight concentration of 188.6; 152.54; 189.07 and 182.75 g/l respectively was determined as described in Example 3. The agitation and aeration rates being reduced from initially at 100 h 800 rpm and 11/min to 550 rpm and 0.75 l/min at ˜400 h.

The residual glucose was <<1 g/l from 25 h and onwards—as determined as described in Example 3.

### “OME17c:

All components are given in g/l.

All components—except for glucose—was heat sterilised together in 40% v/v of the final medium volume at 121° C. for 40 min after pH being adjusted to 5.0 with NaOH/H₃PO₄. Glucose was heat sterilised separately in 60% v/v of the final medium volume and then added to the other components after cooling to below 40° C.

Tap water was used throughout.

| | | |
|---|---|---|
| Tropic Marin ®: | 16.7 | g/l |
| KH₂PO₄ | 10 | g/l |
| “MikroPM” | 40 | g/l |
| “VitaPM” | 40 | g/l |
| Casamino acids, vitamin free | 25.88 | g/l |
| (NH₄)₂SO₄ | 4.3 | g/l |
| Glucose•1H₂O | 284.6 | g/l |

From the above: Y=189 g/l; h=1.37 and f=0.021 at 285 h;
Y=153 g/l; h=1.70 and f=0.021 at 350 h;
Y=189 g/l; h=1.37 and f=0.021 at 450 h,
Y=183 g/l; h=1.42 and f=0.021 at 500 h.

It is to be noted that the variation within the productivity of cells is surprisingly little (when h is constant, Y is also almost constant as illustrated with the results at 285 h and 450 h respectively).

### Example 5

### Lipid Content in Cell Dry Matter from High Cell Density Continuous Cultivations

From the material produced by freeze drying a washed 50 ml broth sample was thoroughly re-suspended in ˜40 ml “½TM”. Lipids were extracted from the re-suspension with chloroform:methanol (2:1 v/v, 0.1% w/v Butylhydroxy toluene (BHT)) and the amount of lipids extracted (g) determined after evaporating all chloroform:methanol. The lipids thus recovered were stored at −80° C. and then subjected to methylation at 40° C. and analysed for DHA according to standard HPLC procedures.

The lipid content in cell dry matter and the polyenoic acid productivity could thus be determined by these methods.

In the fermentation described in Example 3 (residence time ˜30-35 h) the lipid content in cell dry matter at 190 h was determined as (>=) 47.5% w/w.

In the fermentation described in Example 4 (residence time ˜60-70 h) the lipid content in cell dry matter at 350 h (before reducing agitation/aeration) was determined as (>=) 60.1% w/w and the polyenoic acid content 21 (DHA in % w/w of total fatty acids).

In the fermentation described in Example 4 (residence time ˜60-70 h) the lipid content in cell dry matter at 450 h (after reducing agitation/aeration) was determined as (>=) 56.4% w/w and the polyenoic acid content 23 (DHA in % w/w of total fatty acids).

In the fermentation described in Example 4 (residence time ˜60-70 h) the lipid content in cell dry matter at 500 h was determined as (>=) 48.2% w/w and the polyenoic acid content 25 (DHA in % w/w of total fatty acids).

In the fermentation described in Example 4 (residence time ˜60-70 h) the polyenoic acid productivity was thus 0.30, 0.38 and 0.34 g DHA/I/h at 350 h, 450 h and 500 h, respectively. It is to be noted that the variation within the productivity of DHA is surprisingly little.

In conclusion Example 4 demonstrates that it is possible by using the method of the invention to produce high cell concentrations and high DHA concentrations at a residence time of 60-70 h.

1. A method of continuously culturing an auxotrophic marine microorganism under aerobic conditions comprising:
culturing said auxotrophic marine microorganism in a culture medium comprising a carbon source for a residence time of 20 to 100 hours;
adding the carbon source limiting for biomass formation to the culture medium in an amount of Y×h gram per liter of culture medium, wherein h is in the amount of 1.1 to 3.0, and Y is cell dry matter in an amount of 100-300 g/l, wherein biomass is produced and 40% of the biomass produced is made up of components extractable by chloroform:methanol mixtures.

2. The method according to claim 1, wherein the culture medium comprises a nitrogen source in an amount of (Y×h×f) gram per liter of culture medium, wherein f is in the amount of 0.002 to 0.2.

3. The method according to claim 1, wherein the culture medium comprises salts and minerals.

4. The method of claim 1, wherein the culture medium comprises vitamins.

5. The method according to claim 2, wherein h is in the amount of 1.1-2.5.

6. The method according to claim 2, wherein h is in the amount of 1.2-2.0.

7. The method according to claim 2, wherein f is in the amount of 0.004 to 0.1.

8. The method according to claim 2, wherein h is in the amount of 0.01 to 0.04.

9. The method according to claim 1, wherein the auxotrophic marine microorganism is an algae.

10. The method according to claim 1, wherein the auxotrophic marine microorganism is a Thraustochytrids sp.

11. The method according to claim 10, wherein the Thraustochytrids sp. is selected from the group consisting of Schizochytrium and Thraustochytrium.

12. The method according to claim 1, wherein the culturing temperature is 20-35° C.

13. The method according to claim 1, wherein the pH of the culturing medium is 3.0-9.0.

14. The method according to claim 1, wherein chloroform and methanol are mixed in the ratio 2:1 (v/v).

15. The method according to claim 1, wherein a polyenoic acid productivity of at least 0.2 g DHA/I/h is achieved.

16. The method according to claim 1, wherein the residence time of the culture broth in the continuous cultivation process is maintained constant and in the amount of 20-100 h.

17. The method according to claim 1, wherein the residence time of the culture broth in the continuous cultivation process is varied within the amount of 20-100 h.

18. The method according to claim 1, wherein the continuously culturing comprises the following 3 cultivation steps:
a) an initial batch process, followed by
b) a fed batch process, followed by
c) a continuous process.

19. The method according to claim 18, wherein dissolved oxygen is maintained below 10% of saturation from the onset of step c).
